(19) United States
(12) Patent Application Publication
(10) Pub. No.: US 2024/0207812 A1
(43) Pub. Date: Jun. 27, 2024

(54) Cellulose Fibres

(71) Applicant: Speciality Fibres and Materials Limited, London (GB)

(72) Inventors: Graeme Kettlewell, Coventry (GB); Gareth Wynn-Jones, Coventry (GB); Ewa King, Coventry (GB); Simon Martin Finnegan, Coventry (GB)

(73) Assignee: Speciality Fibres and Materials Limited, London (GB)

(21) Appl. No.: 18/437,746

(22) Filed: Feb. 9, 2024

Related U.S. Application Data
(63) Continuation of application No. PCT/GB2022/051982, filed on Jul. 28, 2022.

(30) Foreign Application Priority Data
Oct. 8, 2021 (GB) 2111503

(51) Int. Cl. B01J 20/02; A61L 15/18; A61L 15/28; A61L 15/44; A61L 15/46; B01J 20/04; B01J 20/24; B01J 20/28; B01J 20/30; B01J 20/32

(52) U.S. Cl. CPC B01J 20/0233; A61L 15/18; A61L 15/28; A61L 15/44; B01J 20/04; B01J 20/24; B01J 20/28007; B01J 20/28028; B01J 20/3071; B01J 20/3085; B01J 20/3208; B01J 20/3223; B01J 20/3236; B01J 20/3293; A61L 15/46; A61L 2300/104; A61L 2300/404; A61L 2400/12

(57) ABSTRACT

A method of producing a solution of polymer-coated metal nanoparticles comprising mixing a first aqueous alkaline solution with an aqueous polymer solution to form an aqueous alkaline polymer solution and mixing with an aqueous solution of a metal salt to form a solution of polymer-coated metal nanoparticles. A further method of producing cellulose fibres impregnated with the metal nanoparticles comprises swelling the fibres and mixing the fibres with the solution of polymer-coated metal nanoparticles.

### CROSS REFERENCE TO RELATED APPLICATIONS

The present application is a continuation of PCT Application No. PCT/GB2022/051982, filed Jul. 28, 2022, which claims priority to United Kingdom Patent Application No. 2111503.5, filed Aug. 10, 2021. The contents of these applications are incorporated herein by reference.

### FIELD OF THE INVENTION

The present invention relates to a method for producing metal nanoparticles and impregnating them into cellulose fibres. The invention further relates to the fibres produced thereby and materials and fabrics comprising the fibres.

### BACKGROUND TO THE INVENTION

Fibres useful as components in advanced wound care dressings are known in the art, particularly fibres based on cellulose or cellulose derivatives such as carboxymethylcellulose (CMC), cellulose ethyl sulfonate (CES) and salts thereof. For example, the commercial dressing AQUACEL® (sold by ConvaTec Inc of Skillman, New Jersy, USA) is based on a carboxymethyl cellulose. The commercial dressing URAFIBER® (sold by Smith and Nephew of Hull, United Kingdom) is made from a blend of cellulose fibres (TENCEL®) and CES fibres.

Metals including silver, copper, zinc and mercury are known for their antimicrobial properties. A renewed interest has developed in the use of metallic silver as an antimicrobial agent, especially in wound dressings, driven in part by the development of antibiotic resistant bacteria. Metallic silver is a broad spectrum antibiotic which has been proven to be effective against such resistant bacteria. Current research suggests that due to its mode of action, metallic silver does not allow for the development of bacterial resistance. WO2015/040435 by the present Applicant describes a process for preparing cellulose fibres impregnated with metal nanoparticles.

Wound dressings currently available on the market primarily contain silver in its ionic form i.e. as a salt or other compound. However, the antibacterial properties of these dressings can be short lived due to the solubility of the silver salts or compounds in the aqueous nature of the wound environment, leading to an almost instantaneous and total release from the dressing. The rapid release of ionic silver into a wound could potentially cause toxic effects in host cells as well as bacteria. Some silver salts can also irritate the skin surrounding a wound, and prolonged contact has been reported to cause localised argyria, a permanent grey-blue staining of the skin. Silver salts in general are very sensitive to light, and show rapid and extensive discolouration (turning brown or even black) leading to less than appealing visual characteristics.

One issue with existing attempts to resolve the above problems is that of scalability. Although some processes are effective for small scale production of the fibres, scaling up some processes causes a drop in efficiency and increased costs. It is an object of the present invention to mitigate at least some of the problems described above.

### SUMMARY OF INVENTION

According to a first aspect of the invention, there is provided a method of producing a solution of polymer-coated metal nanoparticles. The method may comprise mixing a first aqueous alkaline solution with an aqueous polymer solution to form an aqueous alkaline polymer solution. The method may comprise mixing the aqueous alkaline polymer solution with an aqueous solution of a metal salt to form a solution of polymer-coated metal nanoparticles.

As used herein, the term “metal nanoparticles” means particles of elemental metal having an average (i.e. mean) diameter of no more than 100 nm.

The first aqueous alkali solution may comprises a Group I hydroxide (e.g. sodium or potassium hydroxide), a Group I carbonate (e.g. Na₂CO₃ or K₂CO₃), a Group I bicarbonate (e.g. NaHCO₃ or KHCO₃), a tetraalkylammonium hydroxide (e.g. tetraethylammonium hydroxide), or mixtures thereof. In a preferred series of embodiments, the first aqueous solution comprises sodium hydroxide and sodium carbonate.

The method according to any one of the preceding claims, wherein the metal salt comprises a metal selected from the group consisting of: silver, copper, zinc, selenium, gold, cobalt, nickel, zirconium, molybdenum, gallium, iron, or any combination thereof. In a preferred series of embodiments, the metal is silver.

The metal salt may be a nitrate, an acetate, a carbonate, a bicarbonate, a sulphate, or mixtures thereof. In a preferred series of embodiments, the metal salt is a nitrate. In a preferred series of embodiments, the metal salt is silver nitrate.

The polymer may be chosen from a group consisting of: a polyamide, polyimide, polyethyleneimine, polyvinylalcohol, pectin, albumin, gelatin, carrageenan, a gum, cellulose or a derivative thereof, poly (N-vinylpyrrolidone), poly (N-vinylcaprolactam), and mixtures thereof. For example, the gum may be xanthan, guar, Arabic, acacia etc. For example, the cellulose derivative may be hydroxyethylcellulose, hydroxypropylcellulose, methylcellulose, hydroxypropylmethylcellulose etc. In a preferred series of embodiments, the polymer is poly (N-vinylpyrrolidone). Poly (N-vinylpyrrolidone) is also known as Povidone, Polyvidone, or PVP.

The polymer may have a weight average molecular weight (M_{w}) of 8 to 360 kg/mol, or from 20 to 80 kg/mol. The polymer may have a M_{w} of more than 10, 15, 20, 25, 30, 32, 34, 36, 38 or 40 kg/mol. The polymer may have a M_{w} of less than 360, 300, 250, 200, 150, 100, 80, 70, 60, 50, 45, 40, 38, 36, 34, 32 or 30 kg/mol. In a series of embodiments, the polymer may have a weight average molecular weight (M_{w}) of 25 to 45 kg/mol, 30 to 40 kg/mol, 32 to 38, or 34 to 36 k/mol.

For example, in a series of embodiments, the polymer is poly (N-vinylpyrrolidone) and wherein the polymer has a weight average molecular weight (M_{w}) of 30 to 40 kg/mol.

In a series of embodiments, the solution of polymer-coated metal nanoparticles is obtainable in the absence of any additional reducing agent.

In step (b), the mixing may be carried out at a temperature of from 20° C. to 120° C. For example, the temperature may be at least 20, 30, 40, 50, 60, 70, 80, 90, 100 or 110. The temperature may be less than 110, 100, 90, 80, 70, 60, 50, 40, or 30° C. In a preferred series of embodiments, the temperature is from 60 to 100° C.

According to a second aspect of the invention, there is provided a solution of polymer-coated metal nanoparticles obtainable by the method described above and herein.

The solution of polymer-coated metal nanoparticles may comprise metal nanoparticles having an average diameter of from 2 to 50 nm. In a preferred series of embodiments, the mean diameter may be from 3 to 12 nm, optionally 4 to 11 nm, 5 to 10, 5 to 9, or 6 to 8 nm. The median diameter may be between 2 and 10 nm, optionally from 3 to 9, 3 to 8, or 4 to 7 nm. The range of nanoparticle diameters within the solution may have a standard deviation of greater than 4, or optionally 4.5.

The solution of polymer-coated metal nanoparticles may comprise metal nanoparticles with a diameter greater than 20 nm, greater than 25 nm, greater than 30 nm, greater than 35 nm or greater than 40 nm. The solution of polymer-coated metal nanoparticles may comprise less than 5% of nanoparticles with diameters greater than 25 nm. Optionally, the solution may comprise from 0.1%, 0.25%, 0.5%, 0.75% or from 1% of nanoparticles, with diameters greater than 25 nm. In some embodiments, the somplution may comprise less than 5%, 4.5%, 4%, 3.5%, 3%, 2.5%, 2%, 1.5% or 1% of nanoparticles with diameters greater than 25 nm.

The solution of polymer-coated metal nanoparticles may comprise metal nanoparticles having a polymer coating with an average thickness of from 40 to 100 nm. Optionally, the polymer-coated metal nanoparticles may have a polymer coating with an average thickness between 50 and 90 nm, 55 to 85 nm, 60 to 80 nm or 65 to 75 nm.

According to a third aspect of the invention, there is provided a method of producing cellulose fibres impregnated with metal nanoparticles. The method may comprise (i) swelling cellulose fibres in a second aqueous alkaline solution to form swollen cellulose fibres. The method may comprise (ii) removing the swollen cellulose fibres from the second aqueous alkaline solution. The method may comprise (iii) mixing the swollen cellulose fibres with a solution of polymer-coated metal nanoparticles so as to impregnate the fibres with the metal nanoparticles. The method may comprise (iv) separating the impregnated cellulose fibres from the solution of polymer-coated metal nanoparticles. The method may comprise (v) optionally washing the impregnated cellulose fibres. The method may comprise (vi) optionally, drying the impregnated cellulose fibres. The solution of polymer-coated metal nanoparticles may be obtainable by the method described above and herein.

The method may comprise preparing the solution of polymer-coated metal nanoparticles according to the method described above and herein.

In one series of embodiments, the impregnated cellulose fibres are dried in step (vi).

The method may comprise, prior to step (v), mixing the impregnated cellulose fibres with the solution of polymer-coated metal nanoparticles so as to impregnate the fibres with the polymer-coated metal nanoparticles. The method may comprise separating the impregnated cellulose fibres from the solution of polymer-coated metal nanoparticles.

In one series of embodiments, in step (iii), the solution of polymer-coated metal nanoparticles is kept at a temperature of from 10 to 30° C. Optionally, the temperature may be from 15 to 25° C.

The second aqueous alkaline solution may comprise a Group I hydroxide, a Group I carbonate, a Group I bicarbonate, a tetraalkylammonium hydroxide, or mixtures thereof.

In one series of embodiments, step (i) comprises incubating the cellulose fibres in the second alkaline solution at a temperature of from 20 to 120° C. Optionally, the temperature may be from 30, 40, 50, 60, 70 or 80° C. to 110, 100, or 95° C. In one series of embodiments, the temperature is from 80 to 100° C.

In one series of embodiments, step (ii) comprises washing the swollen cellulose fibres after their removal from the second aqueous alkaline solution.

In one series of embodiments, the metal nanoparticles are located on both external fibre surfaces and inner fibre pore surfaces.

In one series of embodiments, the impregnated cellulose fibres have a pH of less than 7. Optionally, the impregnated cellulose fibres may have a pH of less than 6 or less than 5.

In one series of embodiments, the metal yield in the cellulose fibres is from 10 to 25%. The metal yield is the proportion of the metal within the nanoparticle solution which is taken up by the fibres. The metal yield may be calculated by experimentally deriving the metal content in the fibres, and dividing it by the amount of metal used to form the nanoparticle solution.

According to a fourth aspect of the invention, there is provided cellulose fibres impregnated with metal nanoparticles obtainable by the method described above and herein.

The cellulose fibres may be impregnated with metal nanoparticles at a metal content of at least 1.5% w/w. The metal content may be based on the weight of the metal within the fibres and the total weight of the cellulose fibres impregnated with metal nanoparticles. Optionally, the metal content may be at least 6% w/w.

The cellulose fibres may be configured such that the average diameter of the metal nanoparticles is from 2 to 50 nm, preferably from 10 to 25 nm. In one series of embodiments, the mean diameter may be from 3 to 12 nm, optionally 4 to 11 nm, 5 to 10, 5 to 9, or 6 to 8 nm. The median diameter may be between 2 and 10 nm, optionally from 3 to 9, 3 to 8, or 4 to 7 nm. The range of nanoparticle diameters within the solution may have a standard deviation of greater than 4, or optionally 4.5.

According to a further aspect of the invention, there is provided an absorbent material comprising a blend of cellulose fibres impregnated with metal nanoparticles as described herein, with at least one other type of fibre.

In some embodiments, the at least one other type of fibre is: a gelling fibre based on alginate, cellulose and modified cellulose, modified chitosan, guar gum, carrageenan, pectin, starch, polyacrylates or copolymers thereof, polyethyleneoxides or polyacrylamides, or mixtures thereof; and/or a non-gelling fibre based on polyester, polyethylene, polyamide, cellulose, thermoplastic bicomponent fibres, glass fibres, or mixtures thereof. In one series of embodiments, the at least one other type of fibre comprises carboxymethyl cellulose (CMC) and lyocell.

The absorbent material may comprise from 0.1 to 10% w/w of metal (based on the total weight of the blended fibres). Optionally, the absorbent material may comprise from 0.1 to 9, 0.2 to 8, 0.3 to 7, 0.4 to 6 or from 0.5 to 5% w/w of metal (based on the total weight of the blended fibres).

According to a further aspect of the invention, there is provided an absorbent article comprising the absorbent material described above and herein. The absorbent article may be a wound care dressing.

### BRIEF DESCRIPTION OF THE FIGURES

Embodiments of the present invention will now be described by way of example and with reference to the accompanying Figures, in which:

FIG. 1 is a graph showing the silver content of the fibres against duration of the dipping time; and

FIG. 2 is a frequency table showing the size distribution of nanoparticles within fibre samples.

### EXAMPLES

### Example 1—Silver Nanoparticles

1a—Silver Nanoparticle Synthesis

Six separate nanoparticle solutions A to F were prepared as follows.
1. 1459 g of deionised (DI) water was placed in a first vessel, such as a 3 L beaker. The first vessel was placed in a water bath set to the temperature in Table 1.
2. 625 g of polyvinylpyrrolidone (PVP) according to Table 1 was added to the beaker gradually with mixing to form a PVP solution.
3. In a second vessel, 0.86 moles of sodium hydroxide and 0.20 moles of sodium carbonate were dissolved in 1096 g of DI water to form a sodium hydroxide and sodium carbonate solution. The second vessel was also placed in the water bath.
4. In a third vessel, a silver nitrate solution was prepared by adding 0.93 moles of AgNO₃ to 371 g of DI water. The third vessel was also placed in the water bath.
5. The first, second, and third vessels were all maintained in the water bath until they had reached the temperature of the water bath.
6 Once the first, second and third vessels had reached the temperature as set out in Table 1 below, the sodium hydroxide and sodium carbonate solution in the second vessel was added to the PVP solution in the first vessel to form an intermediate solution.
7 Subsequently, the silver nitrate solution was added slowly to the intermediate solution and gently stirred. After all of the silver nitrate solution had been added, the reaction was left to run for 20 minutes with constant gentle stirring to yield the silver nanoparticle solutions A to F.
8 The silver nanoparticles in solutions A to F have a coating comprising a polymer shell formed by the PVP.

TABLE 1

| Nano- | | | | | | Nanoparticle |
|---|---|---|---|---|---|---|
| particle | | Mₙ/g | M_{w}/g | | | concentration |
| Solution | PVP | mol⁻¹ | mol⁻¹ | M_{w}/Mₙ | Temperature | (ppm) |
| A | PVP 40 | 14,100 | 35,300 | 2.5 | 90° C. | 7800 |
| | (Sigma | | | | | |
| | Aldrich) | | | | | |
| B | PVP K-15 | 3,900 | 7,300 | 1.9 | 60° C. | 1760 |
| | (Ashland) | | | | | |
| C | PVP K-15 | 3,900 | 7,300 | 1.9 | 90° C. | 1980 |
| | (Ashland) | | | | | |
| D | PVP K-30 | 12,900 | 34,400 | 2.7 | 60° C. | 1140 |
| | (Ashland) | | | | | |
| E | PVP K-30 | 12,900 | 34,400 | 2.7 | 90° C. | 5760 |
| | (Ashland) | | | | | |
| F | Vida-Care C | 3,400 | 19,950 | 5.9 | 90° C. | — |
| | K30P (KCC- | | | | | |
| | Basildon) | | | | | |

1b—Silver Nanoparticle Properties

The silver nanoparticle solutions A to E, and commercially available silver nanoparticles* (PVP AgPURE™—supplied by RAS AG), were analysed by Scanning Transmission Electron Microscopy (STEM) using ImageJ Fiji software to determine the size of the silver core and the PVP coating. The average values are set out in Table 2 below.

The silver nanoparticle solutions A to E, and commercially available silver nanoparticles* (PVP AgPURE™), were tested to determine the minimum bactericidal concentration (MBC). The MBC is the lowest concentration required to kill 99.9% of the bacterium initially inoculated onto an agar plate and is determined by assay and sequential dilution of the bactericidal agent. Typically, a compound is considered bactericidal if the MBC is less than four times the Minimum Inhibitory Concentration. The MBC was determined against Staphylococcus aureus and Pseudomonas aeruginosa and the results set out in Table 2 below.

TABLE 2

| | MBC (ppm) | | Nanoparticle Diameter (nm) | | |
|---|---|---|---|---|---|
| Nanoparticle | Staphylococcus | Pseudomonas | | PVP Shell | Total |
| Solution | aureus | aeruginosa | Ag Core | thickness | diameter |
| A | >4.3 | >4.3 | 18 | 73 | — |
| B | 1.5 | 0.9 | 7 | 46 | 98 |
| C | 7.5 | >15 | 13 | 47 | 106 |
| D | 20.4 | 3.3 | 14 | 76 | 165 |
| E | 8.1 | 6.5 | 12 | 77 | 165 |
| PVP AgPURE ™ | 311 | >311 | 20 | 25 | 70 |

### Example 2—Swelling Cellulose Fibres

2a—Fibre Swelling

Swollen cellulose fibres were produced as follows:
1. 352.8 g of DI water was added to a vessel.
2. Subsequently 57.6 g of 47% NaOH solution was added to the vessel.
3. Subsequently, 39.8 g of Na₂CO₃ was added to the vessel to form a first alkaline solution.
4. 30 g of cellulose fibres (lyocell) were added to the first alkaline solution in the vessel. The vessel containing the cellulose fibres and the alkaline solution was placed in a water bath at 90° C. to effect swelling of the cellulose fibres.
5. The fibres were allowed to swell in the first alkaline solution for 30 minutes.
6. After 30 minutes, the swelled fibres were removed from the first alkaline solution, squeezed to remove excess liquid, and then washed with 500 g of DI water.
7 The washed fibres were removed from the DI water and squeezed to remove excess liquid resulting in washed, swelled cellulose fibres.
   2b—Modified Fibre Swelling

Swollen cellulose fibres were produced as follows:
1. 352.8 g of DI water was added to a vessel.
2. Subsequently, 57.6 g of 47% NaOH solution was added to the vessel to form a second alkaline solution.
3. 30 g of cellulose fibres (lyocell) were added to the second alkaline solution in the vessel to effect swelling of the fibres.
4. The fibres were allowed to swell in the second alkaline solution for 30 minutes at room temperature.
5. After 30 minutes, the swelled fibres were removed from the first alkaline solution, squeezed to remove excess liquid, and then washed with 500 g of DI water.
6. The washed fibres were removed from the DI water and squeezed to remove excess liquid resulting in washed, swelled cellulose fibres.

### Example 3—Cellulose Fibres Impregnated with Silver Nanoparticles

3a—Enhanced Fibre Treatment Process

Four examples (Fibres 1 to 4) of cellulose fibres impregnated with silver nanoparticles were prepared as follows.
1. 1000 ml of a silver nanoparticle solution, produced according to the method of Example 1, was placed in a vessel according to Table 3.
2 Subsequently, 60 g of washed, swelled, not dried, cellulose fibre, produced according to the method of Example 2a, was added to the vessel.
3. The vessel containing the silver nanoparticle solution and the fibres was heated at 90° C. for 2.5 hrs, forming silver nanoparticle impregnated fibres.
4. After 2.5 hours, the impregnated fibres were removed from the vessel and squeezed to remove excess liquid. The impregnated fibres were placed in a new vessel.
5. In a separate vessel, a citric acid solution was formed by dissolving 40 g of citric acid monohydrate in 860 g of DI water.
6. The citric acid solution was added to the vessel containing the impregnated fibres. The citric acid solution and impregnated fibres were heated at 90° C. for 30 minutes.
7 After 30 minutes, the fibres were removed from the vessel and squeezed to remove excess liquid.
8. The fibres were then placed in a new vessel and washed twice in 900 g of DI water. After washing, the fibres were removed from the vessel and squeezed to remove excess liquid. The fibres were finally washed with 450 g of acetone and then dried in an oven at 60° ° C. to form Fibres 1 to 4.

TABLE 3

| Fibre Sample | Nanoparticle solution used |
|---|---|
| Fibres 1 | A |
| Fibres 2 | C |
| Fibres 3 | E |
| Fibres 4 | F |

3b—Dipping Fibre Treatment Process

Three examples (Fibres 5 to 7) of cellulose fibres impregnated with silver nanoparticles were prepared as follows.
1. 800 ml of a silver nanoparticle solution, produced according to the method of Example 1, was placed in a vessel according to Table 4.
2 Subsequently, 60 g of washed, swelled, dried cellulose fibre, produced according to the method of Example 2a, was added to the vessel.
3. The fibres were left in the vessel containing the silver nanoparticle solution for two minutes at room temperature, forming impregnated fibres.
4. The impregnated fibres were removed from the vessel and squeezed to remove excess liquid. The excess liquid was returned to the vessel containing the silver nanoparticle solution.
5. The squeezed fibres were dried in an oven at 90° C. for 20 minutes.
6 The dipping process was repeated by returning the dried, impregnated fibres to the vessel containing the silver nanoparticle solution and left for a further two minutes at room temperature.
7. The impregnated fibres were removed from the silver nanoparticle solution and placed in a new vessel.
8 The impregnated fibres were then washed twice in 500 g of DI water. After washing, the fibres were removed from the vessel and squeezed to remove excess liquid. The fibres were finally washed with 450 g of acetone and 4 g of Tween™ 20 (Sigma Aldrich).
9. Subsequently, the washed fibres were dried in an oven at 60° C. to form Fibres 5 to 7.

TABLE 4

| Fibre Sample | Nanoparticle solution used |
|---|---|
| Fibres 5 | A |
| Fibres 6 | C |
| Fibres 7 | E |

3c—Dipping Fibre Treatment Process Using Modified Fibre Swelling

Three examples (Fibres 8 to 10) of cellulose fibres impregnated with silver nanoparticles were prepared as follows.

The process of Example 3b was repeated, except that the swelled cellulose fibres used were produced according to the method of Example 2b. The nanoparticle solutions used were those set out in Table 5 below. The process formed Fibres 8 to 10.

TABLE 5

| Fibre Sample | Nanoparticle solution used |
|---|---|
| Fibres 8 | A |
| Fibres 9 | C |
| Fibres 10 | E |

3d—Dipping Fibre Treatment Process Using Non-Swollen Fibres

Three examples (Fibres 11 to 13) of cellulose fibres impregnated with silver nanoparticles were prepared as follows.
1. 800 ml of a silver nanoparticle solution, produced according to the method of Example 1, was placed in a vessel according to Table 6.
2. Subsequently, 60 g of cellulose fibre (lyocell) that had not been previously swollen was added to the vessel.
3 The fibres were left in the vessel containing the silver nanoparticle solution for two minutes at room temperature, forming impregnated fibres.
4 The impregnated fibres were removed from the vessel and squeezed to remove excess liquid. The excess liquid was returned to the vessel containing the silver nanoparticle solution.
5. The squeezed fibres were dried in an oven at 90° C. for 20 minutes.
6 The dipping process was repeated by returning the dried, impregnated fibres to the vessel containing the silver nanoparticle solution and left for a further two minutes at room temperature.
7 The impregnated fibres were removed from the silver nanoparticle solution and placed in a new vessel.
8 The impregnated fibres were then washed twice in 500 g of DI water. After washing, the fibres were removed from the vessel and squeezed to remove excess liquid. The fibres were finally washed with 450 g of acetone and 4 g of Tween™ 20 (Sigma Aldrich).
9 Subsequently, the washed fibres were dried in an oven at 60° ° C. to form Fibres 11 to 13.

TABLE 6

| Fibre Sample | Nanoparticle solution used |
|---|---|
| Fibres 11 | A |
| Fibres 12 | C |
| Fibres 13 | E |

The above processes are summarised in Table 7 below.

TABLE 7

| | Nanoparticle | Fibre Swelling | NP Impregnation |
|---|---|---|---|
| Fibre Sample | solution | Process | Process |
| Fibres 1 | A | Ex 2a | Enhanced (Ex 3a) |
| Fibres 2 | C | Ex 2a | Enhanced (Ex 3a) |
| Fibres 3 | E | Ex 2a | Enhanced (Ex 3a) |
| Fibres 4 | F | Ex 2a | Enhanced (Ex 3a) |
| Fibres 5 | A | Ex 2a | Dipping (Ex 3b) |
| Fibres 6 | C | Ex 2a | Dipping (Ex 3b) |
| Fibres 7 | E | Ex 2a | Dipping (Ex 3b) |
| Fibres 8 | A | Ex 2b | Dipping (Ex 3c) |
| Fibres 9 | C | Ex 2b | Dipping (Ex 3c) |
| Fibres 10 | E | Ex 2b | Dipping (Ex 3c) |
| Fibres 11 | A | N/A | Dipping (Ex 3d) |
| Fibres 12 | C | N/A | Dipping (Ex 3d) |
| Fibres 13 | E | N/A | Dipping (Ex 3d) |

### Example 4—Determination of Particle Size

The size of the silver nanoparticles impregnated within the Fibres 1 to 13 was measured as follows and the results set out in Table 8.
1. A sample of Fibres 1 was mixed with an epoxy resin. The epoxy resin was formed from Araldite CY212 plus dodecenylsuccinic anhydride (DDSA) and one drop per ml of benzyldimethylamine (BDMA).
2 The fibres and epoxy resin mixture was subsequently cured in an oven at 60° C. for 36-72 hours to form resin embedded fibres.
3. A sample of the resin embedded fibres was taken by sectioning the resin embedded fibres using a Leica UC 6 Ultra microtome with a diamond knife at 85-90 nm. The sample was placed onto a 200 mesh coated copper grid. The sample was viewed on a FEI Tenai TEM at 80 Kv operating voltages and images recorded using Gatan Digital Micrograph software. The diameter of the silver nanoparticle core within the PVP coating was measured using ImageJ Fiji software.
4. The process was repeated for each of the Fibres 2 to 13.

TABLE 8

| | Ag nanoparticle core |
|---|---|
| | diameter (nm) |
| Fibres 1 | 18 |
| Fibres 2 | 15 |
| Fibres 3 | 23 |
| Fibres 4 | 12 |
| Fibres 5 | 4 |
| Fibres 6 | 5 |
| Fibres 7 | 8 |
| Fibres 8 | 5 |
| Fibres 9 | 6 |
| Fibres 10 | 5 |
| Fibres 11 | 6 |
| Fibres 12 | 5 |
| Fibres 13 | 5 |

### Example 5—Determination of Silver Content

The silver content of Fibres 1 to 13 was determined as follows and the results set out in Table 9 below.
1. A sample of Fibres 1 was placed in a vessel.
2. A solution of nitric acid was added to the vessel to dissolve the silver within the fibres, forming a silver solution.
3. The silver solution was titrated against potassium thiocyanate using a ferric alum sulphate indicator. When the ferric alum sulphate indicator showed a reddish brown tint, the titration was ended.
4. The silver content was then calculated from the amount of potassium thiocyanate used by calculating the weight of the silver determined from the titration, divided by the starting weight of the fibres using the equation:

$$\% silver = \frac{titre \times 1.08}{weight of sample ( g )}$$

The silver content determined is set out in Table 9 below.

TABLE 9

| | Ag % w/w |
|---|---|
| Fibres 1 | 8.3 |
| Fibres 2 | 7.4 |
| Fibres 3 | 6.9 |
| Fibres 4 | 8.4 |
| Fibres 5 | 6.4 |
| Fibres 6 | 0.9 |
| Fibres 7 | 1.3 |
| Fibres 8 | 7.3 |
| Fibres 9 | 0.9 |
| Fibres 10 | 1.1 |
| Fibres 11 | 2.3 |
| Fibres 12 | 3.8 |
| Fibres 13 | 1.0 |

### Example 6—Dipping Time

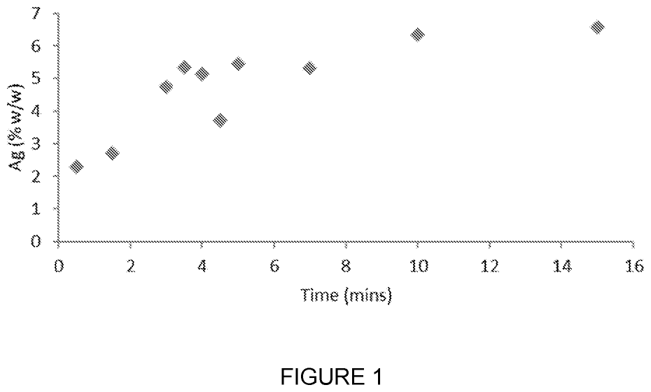

The effect of the dipping time was investigated as follows and the results set out in Table 10 below.
1. Swollen cellulose fibres were prepared according to the method of Example 2b above.
2 The swollen cellulose fibres were then impregnated with silver nanoparticles according to the method described in Example 3b and using Nanoparticle solution A.
3. The method of Example 3b was changed by varying the dipping time. The dipping time is the total length of time the swollen cellulose fibres were kept in the nanoparticle solution i.e. in steps 3 and 6 of Example 3b.
4. The process was repeated and the dipping time varied according to Table 10.
5. The silver content of the fibres was recorded and is shown in Table 10 and FIG. 1.

TABLE 10

| Experiment | Dipping | Ag content |
|---|---|---|
| No. | time (mins) | (% w/w) |
| 6.1 | 0.5 | 2.3 |
| 6.2 | 1.5 | 2.7 |
| 6.3 | 3 | 4.8 |
| 6.4 | 3.5 | 5.4 |
| 6.5 | 4 | 5.2 |
| 6.6 | 4.5 | 3.7 |
| 6.7 | 5 | 5.5 |
| 6.8 | 7 | 5.3 |
| 6.9 | 10 | 6.4 |
| 6.10 | 15 | 6.6 |

### Example 7—Manufacture of Gel-Forming Fabric Containing Silver Nanoparticles

A gel-forming fabric containing silver nanoparticles was prepared according to the method below.
1. Fibres 5 were cut to short lengths of approximately 50 mm.
2. Additional lyocell fibres, which had not been swollen or impregnated with silver nanoparticles, were cut to the same approximate length of 50 mm.
3. A sample of gel-forming carboxymethyl cellulose (CMC) fibres (SFM Limited) were also cut to an approximate length of 50 mm.
4. The cut Fibres 5, lyocell fibres, and CMC fibres were then blended using standard non-woven carding equipment. 14 g of Fibres 5, having a silver content of 6.4%, was blended with 60 g of the CMC fibres and 26 g of the lyocell fibres to achieve a blend of fibres which comprised 60% w/w of gelling fibres and 40% w/w of non-gelling fibres. 5. The blended fibres were then needle bonded for form a silver nanoparticle containing gel-forming fabric of 200 gsm and having a silver content of 18 mg/100 cm².

A range of fabrics can be produced using the above process which have different silver contents by adjusting the ratio of silver-containing fibres (i.e. Fibres 5) and silver-free fibres (i.e. the non-impregnated lyocell fibres and the CMC fibres) and/or the silver content of the silver-containing fibres. For example, in a hypothetical example using a silver nanoparticle impregnated fibre having a silver content of 3.2%, this could comprise 47 g of the silver nanoparticle impregnated fibres, 60 g of CMC fibres, and 3 g of lyocell fibres. The fibres would thus be blended in a ratio of 50:50% w/w of gelling and non-gelling fibres. When carded and needle bonded into a 120 gsm fabric, the overall silver content of the fabric would be 18 mg/100 cm².

The thickness/density of a fabric is usually measured according to the weight per unit of area, typically grams per square metre (gsm). The fabric thickness and/or density of the silver nanoparticle containing gel-forming fabric can be adjusted by adjusting the operating parameters of the textile equipment in the manner known to one skilled in the art. For example, the weight of fibres fed into the card, the speed of the take-up belts and the cross folder can all be altered in order to modify the desired output. In two examples, a silver nanoparticle containing gel-forming fabric was prepared at 120 gsm (Fabric 14) and at 200 gsm (Fabric 15).

### Example 8—Silver Released from Non-Woven Fabrics

The amount of silver released by the fabrics produced the process described in Example 7 was investigated and compared to commercially available silver-containing fabrics. The results are set out in Table 11 below.
1. 50 ml of distilled water was added to a 100 ml flask.
2. A 25 cm² sample of Fabric 14 was added to the flask containing distilled water. The flask was covered to prevent evaporation and incubated at 37° C. with agitation at 40 rpm.
3. After 5 minutes 1.0 ml of liquid was removed from the flask. 1.0 ml of fresh distilled water added to the flask.
4. The 1 ml of liquid removed from the flask was tested to determine the silver concentration in the liquid by Inductively Coupled Plasma-Optical Emission Spectrometry (ICP-OES).
5. Steps 3 and 4 were repeated after 10 minutes, 30 minutes, 1 hour and 5 hours and the silver concentration recorded in Table 11.
6. The steps 1 to 5 above were repeated for Fabric 15 and Comparison fabrics 16 and 17.

TABLE 11

| | | Silver Concentration (ppm) | | | | |
|---|---|---|---|---|---|---|
| | | 5 | 10 | 30 | 1 | 5 |
| Test Sample | Product | mins | mins | mins | hour | hours |
| Fabric 14 | Fabric 14 | 2.3 | 2.9 | 4.9 | 6.2 | 12.0 |
| | (120 gsm) | | | | | |
| Fabric 15 | Fabric 15 | 1.6 | 3.6 | 5.4 | 6.6 | 13.3 |
| | (200 gsm) | | | | | |
| Comparison | Aquacel Ag | 12.1 | 15.0 | 21.2 | 22.3 | 26.0 |
| fabric 16 | Extra | | | | | |
| Comparison | Kerracel Ag | 12.7 | 15.4 | 21.7 | 24.2 | 26.7 |
| fabric 17 | | | | | | |

Aquacel™ Ag Extra is a carboxymethylcellulose fabric containing ionic silver, supplied by Convatec™ (Reading, UK). Kerracel™ Ag is a carboxymethylcellulose dressing with silver oxysalts, supplied by 3M (Saint Paul, Minnesota, USA).

### Example 9—Determination of Fabric Antibacterial Efficacy

The antibacterial properties of the gel-forming fabrics containing silver nanoparticles were investigated as set out below.
1. Six gel-forming fabrics containing silver nanoparticles were prepared according to the method of Example 7, except that Fibres 8 were used instead of the Fibres 5.
2. The six fabrics were produced to different fabric weights as set out in Table 12 below. The silver content of the fabrics was achieved by varying the relative proportion of Fibres 8 compared to the lyocell and CMC fibres to form Fabrics 18 to 23.
3. The antibacterial properties of each of Fabrics 18 to 23 was evaluated using a modified AATCC-100 test method:
   A test sample of 17.6 cm² of each Fabric 18 to 23 was sterilised using gamma irradiation. Each of the sterilised test samples were subsequently saturated with a simulated wound fluid such as those known to one skilled in the art. Each of the saturated test samples was incubated at 37° C. for four days and subsequently inoculated with 1×10⁻⁶ cfu (colony forming unit) of bacteria. The inoculated fabric was then incubated undisturbed in a sealed jar for 24 hours. After 24 hours viable bacteria were recovered, enumerated and a log reduction calculated.

TABLE 12

| Test | Fabric | Ag (mg/ | | Escherichia | Klebsiella |
|---|---|---|---|---|---|
| Sample | density | 100 cm)² | MRSA | Coli | pneumoniae |
| Fabric 18 | 120 gsm | 18 | 6.2 | 6.0 | 6.0 |
| Fabric 19 | 160 gsm | 18 | 4.7 | 4.5 | 6.4 |
| Fabric 20 | | 24 | 6.6 | 4.6 | 5.0 |
| Fabric 21 | 200 gsm | 18 | 5.1 | 5.0 | 6.3 |
| Fabric 22 | | 24 | 6.8 | 6.5 | 5.7 |
| Fabric 23 | | 30 | 6.8 | 6.5 | 6.4 |
| Aquacel | 180 gsm | 22 | 4.6 | 5.9 | 4.8 |
| Ag Extra | | | | | |

### Example 10—Nanoparticle Molecular Weight Versus Minimum Bactericidal Concentration

The minimum bactericidal concentration of the nanoparticles solutions was investigated as follows.
1. Four silver nanoparticle solutions were prepared according to Table 13 below. The method was the same as described in Example 1, except that the PVP used was taken from Table 13, and the water bath temperature was set at 90° C., to produce Nanoparticle solutions A and G to I.

TABLE 13

| Nanoparticle | | | | | |
|---|---|---|---|---|---|
| Solution | PVP | Mₙ/g mol⁻¹ | M_{w}/g mol⁻¹ | M_{w}/Mₙ | Temperature |
| A | PVP 40 (Sigma Aldrich) | 14,100 | 35,300 | 2.5 | 90° C. |
| G | PVP 10 (Sigma Aldrich) | 4,700 | 11,800 | 2.5 | 90° C. |
| H | PVP 29 (Sigma Aldrich) | 9,900 | 25,400 | 2.6 | 90° C. |
| I | PVP 55 (Sigma Aldrich) | 13,100 | 32,900 | 2.5 | 90° C. |

The Minimum Bactericidal Concentration (MBC) of silver nanoparticle solutions A and G to I was determined and set out in Table 14 below.

TABLE 14

| | | Nanoparticle solution | | | |
|---|---|---|---|---|---|
| | | A | G | H | I |
| Lambda max | | 414 | 418 | 405 | 409 |
| Agglomeration ratio | | 3.5 | 2.8 | 2.2 | 2.0 |
| Concentration (ppm) | | 10,200 | 11,960 | 4,520 | 2,420 |
| Minimum | S. aureus | 0.6 | 0.7 | 0.3 | 0.2 |
| Bactericidal | 6539 | | | | |
| Concentration | E. coli | 1.2 | 2.9 | 1.1 | 0.6 |
| (ppm) | 8739 | | | | |

Nanoparticle solutions A (10,200 ppm) and G (11,960 ppm) produced the largest concentration of silver nanoparticles in solution. The analysis of the nanoparticle solutions was carried out with the use of Analytic Jena Specord 205 spectrophotometer.

The UV-VIS analysis consisted of acquiring lambda max (the wavelength corresponding to the highest absorbency), agglomeration ratio (the intensity of the absorbance at lambda max divided by the intensity of the absorbance at ca. 500 nm) and concentration of nanoparticles. Nanoparticle solution G had the largest silver nanoparticles, λₘₐₓ=418 nm. Nanoparticle solution A had the lowest amount of agglomerated silver nanoparticles, evidenced by the agglomeration ratio (ratio of λₘₐₓ absorbance at 400-410 nm and absorbance at 500 nm). Nanoparticle solution I has the lowest Minimum Bactericidal Concentration (0.15 ppm versus Staphylococcus aureus and 0.58 ppm versus Escherichia Coli). Without wishing to be unduly bound by theory, it is desirable to have a high nanoparticle concentration to maximise efficiency of the process and to avoid silver wastage. A low Minimum Bactericidal Concentration is desirable to maximise the antibacterial effect of the nanoparticles. A high agglomeration ratio (a low amount of agglomerated particles compared to individual particles) is desired because agglomerated particles have a smaller surface area per unit mass than smaller individual nanoparticles and their antibacterial performance is understood to be improved because of this.

### Example 11—Odour Control

The ability of the gel-forming fabrics containing silver nanoparticles to control odour was investigated as follows.

A gel-forming fabric containing silver nanoparticles (Fabric 24) was prepared according to the method of Example 7, except using the Fibres 8 instead of Fibres 5. Fabric 24 was then compared against four commercially available dressings as set out in Table 15.

The tests were carried out by the Surgical Materials Testing Laboratory (Cardiff, GB) according to SMTL test method TM-283. The method used is as follows:
1. A test sample of a fabric according to Table 15 was placed over a recess in a stainless steel plate and covered with a Perspex™ dome. A 50 ml syringe attached to a syringe driver was filled with a 2% diethylamine solution.
2. The 2% diethylamine solution was then infused onto the test sample through the recess via the syringe driver which was set at an infusion rate of 30 ml/hour.
3. The time taken for a gas analyser to detect a diethylamine concentration of 15 ppm was recorded.
4. The volume of test solution that had been applied to the dressing was calculated.
5. Testing was performed on 3 replicates.

TABLE 15

| | | | Volume of |
|---|---|---|---|
| | | | diethylamine |
| | | | in ml |
| Test Sample | Product | Description | (Std dev) |
| Fabric 24 | Fabric 24 | Gel-forming fabric comprising | 1.4 (0.6) |
| | | Fibres 8, lyocell and | |
| | | CMC; 200 gsm | |
| Comparison | Suprasorb | Calcium alginate dressing | 0.8 (0.1) |
| fabric 25 | A + Ag⁽¹⁾ | with ionic silver | |
| Comparison | Aquacel Ag | CMC dressing with | 0.9 (0.1) |
| fabric 26 | Extra⁽²⁾ | ionic silver. | |
| Comparison | Vliwaktiv⁽¹⁾ | Absorbent dressing with | 1.7 (0.1) |
| fabric 27 | | an odour adsorbent layer. | |
| Comparison | Vliwaktiv | Absorbent dressing with | 4.0 (0.4) |
| fabric 28 | Ag⁽¹⁾ | an odour adsorbent layer | |
| | | and silver. | |

⁽¹⁾Lohmann and Rauscher (Rengsdorf, Germany)
⁽²⁾Convatec (Reading UK)

### Example 12—Drying

The importance of the drying step in preparing the silver nanoparticle-containing fibres was investigated as follows.
1. Two samples of Fibres 8 were prepared as follows:
2. A first 250 g sample of Fibres 8 was prepared according to Example 3c above, scaled up accordingly. The drying steps during the dipping process were all carried out in an oven which held eight other batches of fibres being dried. The atmosphere within the oven thus had a high relative humidity (e.g. above 25%), forming Fibres 8a.
3. A second 250 g sample of Fibres 8 was prepared according to Example 3c above, scaled up accordingly. The drying steps during the dipping process were all carried out in an oven wherein the humidity was immediately removed from the oven using an extraction fan, forming Fibres 8b.
4. Two gel-forming fabrics comprising silver nanoparticles were produced according to Example 7, except that the first fabric used the Fibres 8a and the second fabric used Fibres 8b.
5. The antibacterial efficacy of the two fabrics was determined according to the method of Example 9 and the results recorded in Table 16.

TABLE 16

| | Fibres 8a | Fibres 8b |
|---|---|---|
| | High humidity | Low humidity |
| Relative humidity | (average 27.6%) | (average 10.8%) |
| MRSA | 3.5 | 5.1 |
| K. pneumoniae | 0.0 | 5.3 |
| P. aeruginosa | 3.3 | 4.1 |
| E. coli | 3.1 | 5.0 |

### Example 13—Comparison to WO2015/040435A1

1. A first sample of Fibres 8 was prepared according to method of Example 3c.
2 A second sample of fibres were prepared according to the method of Example 1.1a described in WO2015/040435A1 (Comparative Fibres 29). Comparative Fibres 29 are cellulose fibres impregnated with silver nanoparticles.
3 The silver content of the Fibres 8 and Comparative Fibres 29 were measured according to the method described in Example 5.
4. The silver yield was then calculated on a mass balance basis. The silver yield is the mass of silver present in the fibres divided by the total mass of silver present within the silver nitrate used to form the fibres, expressed as a percentage. A silver yield of 100% indicates that all the silver within the silver nitrate has been taken up by the fibres as silver nanoparticles. The silver yield was calculated across multiple repeat samples of Fibres 8 which produced the range shown in Table 17.

TABLE 17

| | Comparative | |
|---|---|---|
| | Fibres 29 | Fibres 8 |
| Silver content (% w/w) | 1.0 | 4.0 |
| Silver yield (as % of Ag added) | <2% | 10-25% |

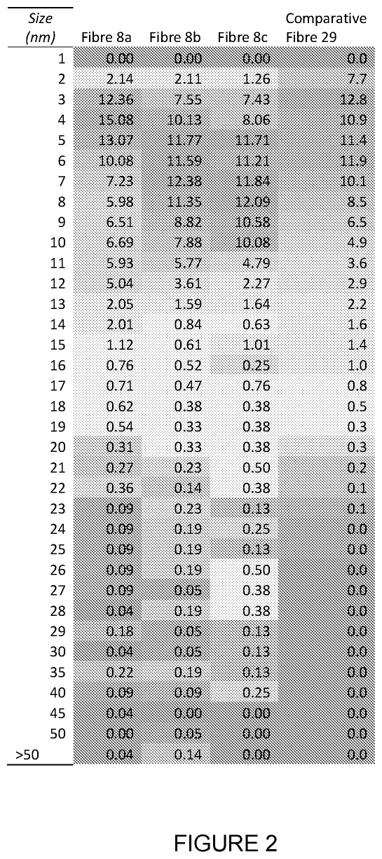

Three further samples of Fibres 8 (Fibres 8a-c) and the Comparative Fibres 29 were tested to investigate the size distribution of the silver nanoparticles within the fibres samples. Testing was carried out using STEM and ImageJ Fiji software to measure the nanoparticles observed, and the total numbers of particles at each size was counted and plotted in the frequency table in FIG. 2. The values are shown as a percentage of the total number of nanoparticles counted. Averages were calculated for the data as set out in Table 18 below.

TABLE 18

| | | | | Comparative |
|---|---|---|---|---|
| | Fibre 8a | Fibre 8b | Fibre 8c | Fibre 29 |
| Median/nm | 5 | 6 | 6 | 5 |
| Mean/nm | 7.5 | 6.71 | 8.1 | 6.8 |
| Mean² | 55.7 | 45.0 | 65.4 | 46.6 |
| Standard | 4.7 | 5.9 | 4.7 | 3.8 |
| deviation | | | | |

Without wishing to be bound by theory, it is understood that the nanoparticles in the Fibres 8a-c were less homogenous than those in Comparative Fibres 29, which were tightly clustered between 2 and 10 nm and the largest observed nanoparticles at 23 nm. In contrast, the average nanoparticle diameter was observed to be greater in Fibres 8a-c, with the majority of nanoparticles being in the 3 nm to 12 nm range, and a small proportion of particles up to 50 nm in diameter. The standard deviation for Fibres 8a-c was larger than that for Comparative Fibre 29, showing a broader spread of nanoparticles sizes. It is believed that the decreased homogeneity and the presence of larger nanoparticles contributes to the continuing efficacy of Fibres 8 over time compared to existing fibres.

### Example 14—Cytotoxicity

A sample of Fabric 24 was prepared according to the method of Example 10 using Fibres 8 with a ratio of 60:40 of gelling to non-gelling fibres. The proportion of the silver containing fibres within the non-gelling fibres portion was selected to achieve a fabric with a silver content of 18 mg/100 cm². The cytotoxicity of the Fabric 24 was tested by NAMSA according to the method of ISO10993-5.

The test was repeated for an existing silver-containing fabric. Comparison Fabric 29 is a calcium alginate material containing ionic silver, produced by the present Applicant.

TABLE 19

| | | Cell |
|---|---|---|
| Test sample | Product | viability (%) |
| Fabric 24 | Fabric 24 | 85% |
| Comparison | Calcium alginate | 5% |
| fabric 29 | fabric containing | |
| | ionic silver (SFM) | |

Cell viability for Fabric 24 was higher than the comparison fabric, indicating a lower in vitro cytotoxicity.

1. A method of producing a solution of polymer-coated metal nanoparticles, the method comprising:
a) mixing a first aqueous alkaline solution with an aqueous polymer solution to form an aqueous alkaline polymer solution; and
b) mixing the aqueous alkaline polymer solution with an aqueous solution of a metal salt to form a solution of polymer-coated metal nanoparticles.

2. The method according to claim 1, wherein the first aqueous alkali solution comprises a Group I hydroxide, a Group I carbonate, a Group I bicarbonate, a tetraalkylammonium hydroxide, or mixtures thereof; preferably wherein the first aqueous solution comprises sodium hydroxide and sodium carbonate.

3. The method according to any one of the preceding claims, wherein the metal salt comprises a metal selected from the group consisting of: silver, copper, zinc, selenium, gold, cobalt, nickel, zirconium, molybdenum, gallium, iron, or any combination thereof; preferably wherein the metal is silver.

4. The method according to any one of the preceding claims, wherein the metal salt is a nitrate, an acetate, a carbonate, a bicarbonate, a sulphate, or mixtures thereof; preferably wherein the metal salt is a nitrate.

5. The method according to any one of the preceding claims, wherein the polymer is chosen from a group consisting of: a polyamide, polyimide, polyethyleneimine, polyvinylalcohol, pectin, albumin, gelatin, carrageenan, gum, cellulose or a derivative thereof, poly (N-vinylpyrrolidone), poly (N-vinylcaprolactam), and mixtures thereof; preferably wherein the polymer is poly (N-vinylpyrrolidone).

6. The method according to any one of the preceding claims, wherein the polymer has a weight average molecular weight (M_{w}) of 20 to 80 kg/mol.

7. The method according to claims 5 and 6, wherein the polymer is poly (N-vinylpyrrolidone) and wherein the polymer has a weight average molecular weight (M_{w}) of 30 to 40 kg/mol.

8. The method according to any one of the preceding claims, wherein the solution of polymer-coated metal nanoparticles is obtainable in the absence of any additional reducing agent.

9. The method according to any one of the preceding claims, wherein in step (b), the mixing is carried out at a temperature of from 20 to 120° C., preferably from 60 to 100° C.

10. A solution of polymer-coated metal nanoparticles obtainable by the method of any one of the preceding claims.

11. The solution of polymer-coated metal nanoparticles according to claim 10, wherein the metal nanoparticles have an average diameter of from 2 to 50 nm, preferably from 3 to 12 nm.

12. The solution of polymer-coated metal nanoparticles according to either claim 10 or 11, wherein the metal nanoparticles have a polymer coating with an average thickness of from 40 to 100 nm.

13. A method of producing cellulose fibres impregnated with metal nanoparticles, the method comprising:
(i) swelling cellulose fibres in a second aqueous alkaline solution to form swollen cellulose fibres;
(ii) removing the swollen cellulose fibres from the second aqueous alkaline solution;
(iii) mixing the swollen cellulose fibres with a solution of polymer-coated metal nanoparticles so as to impregnate the fibres with the metal nanoparticles;
(iv) separating the impregnated cellulose fibres from the solution of polymer-coated metal nanoparticles;
(v) optionally washing the impregnated cellulose fibres; and
(vi) optionally, drying the impregnated cellulose fibres,
wherein the solution of polymer-coated metal nanoparticles is obtainable by the method of any one of claims 1 to 9 or is according to any one of claims 10 to 12.

14. The method according to claim 13, comprising preparing the solution of polymer-coated metal nanoparticles according to the method of any one of claims 1 to 9.

15. The method according to any one of claims 13 to 14, wherein the impregnated cellulose fibres are dried in step (vi).

16. The method according to claim 15, comprising, prior to step (v), mixing the impregnated cellulose fibres with the solution of polymer-coated metal nanoparticles so as to impregnate the fibres with the polymer-coated metal nanoparticles; and separating the impregnated cellulose fibres from the solution of polymer-coated metal nanoparticles.

17. The method according to any one of claims 13 to 16, wherein in step (iii), the solution of polymer-coated metal nanoparticles is kept at a temperature of from 10 to 30° C., preferably 15 to 25° C.

18. The method according to any one of claims 13 to 17, wherein the second aqueous alkaline solution comprises a Group I hydroxide, a Group I carbonate, a Group I bicarbonate, a tetraalkylammonium hydroxide, or mixtures thereof.

19. The method according to any one of claims 13 to 18, wherein step (i) comprises incubating the cellulose fibres in the second alkaline solution at a temperature of from 20 to 120° C., preferably 60 to 100° C.

20. The method according to any one of claims 13 to 19, wherein step (ii) comprises washing the swollen cellulose fibres after their removal from the second aqueous alkaline solution.

21. The method according to any one of claims 13 to 20, wherein the metal nanoparticles are located on both external fibre surfaces and inner fibre pore surfaces.

22. The method according to any one of claims 13 to 21, wherein the impregnated cellulose fibres have a pH of less than 7.

23. The method according to any one of claims 13 to 22, wherein the metal yield in the cellulose fibres is from 10 to 25%.

24. Cellulose fibres impregnated with metal nanoparticles obtainable by the method of any one of claims.

25. The cellulose fibres according to claim 24, impregnated with metal nanoparticles at a metal content of at least 1.5% w/w (based on the weight of the metal and the total weight of the cellulose fibres impregnated with metal nanoparticles), preferably, at least 6% w/w (based on the weight of the metal and the total weight of the cellulose fibres impregnated with metal nanoparticles).

26. The cellulose fibres according to any one of claims 24 to 25, wherein the average diameter of the metal nanoparticles is from 2 to 50 nm, preferably from 10 to 25 nm.

27. An absorbent material comprising a blend of cellulose fibres impregnated with metal nanoparticles according to any one of claims 24 to 26, with at least one other type of fibre.

28. The absorbent material according to claim 27, wherein the at least one other type of fibre is:
a gelling fibre based on alginate, cellulose and modified cellulose, modified chitosan, guar gum, carrageenan, pectin, starch, polyacrylates or copolymers thereof, polyethyleneoxides or polyacrylamides, or mixtures thereof; and/or
a non-gelling fibre based on polyester, polyethylene, polyamide, cellulose, thermoplastic bicomponent fibres, glass fibres, or mixtures thereof.

29. The absorbent material of claim 28, wherein the at least one other type of fibre comprises carboxymethyl cellulose (CMC) and lyocell.

30. The absorbent material according to any one of claims 27 to 29, comprising from 0.1 to 10% w/w of metal (based on the total weight of the blended fibres), and preferably from 0.5 to 5% w/w of metal (based on the total weight of the blended fibres).

31. An absorbent article comprising the absorbent material of any one of claims 27 to 30.

32. The absorbent article according to claim 31, wherein the absorbent article is a wound care dressing.
